(19) United States
(12) Patent Application Publication
(10) Pub. No.: US 2008/0154344 A1
(43) Pub. Date: Jun. 26, 2008

(54) System and method for treating benign prostatic hyperplasia

(76) Inventors: Robert M. Trusty, Cincinnati, OH (US); Victor C. Esch, Albuquerque, NM (US); Richard Rox Anderson, Boston, MA (US)

Correspondence Address: Christopher W. Elswick, 2000 Courthouse Plaza, N.E., P.O. Box 8801, Dayton, OH, 45401-8801, US

(21) Appl. No.: 11/645,144

(22) Filed: Dec. 22, 2006

(51) Int. Cl. A61N 5/06

(52) U.S. Cl. 607/92; 607/89; 607/93

(57) ABSTRACT

A method for treating benign prostatic hyperplasia using a laser is provided. The method includes emitting, in proximity to prostatic tissue, laser light at a wavelength that is controlled to be within at least one of (i) a range between about 1275 nm and about 1475 nm or (ii) a range between about 1830 nm and about 2010 nm. The wavelength is selected to have a higher absorption by water than laser light at a wavelength of 830 nm and a lower absorption by hemoglobin than laser light at the wavelength of 830 nm. Emission of the laser light is controlled such that the prostatic tissue is heated to a temperature of less than about 100° C. to coagulate the prostatic tissue.

### TECHNICAL FIELD

The present application relates generally to treatment of benign prostatic hyperplasia using a laser.

### BACKGROUND

Surgeons frequently employ medical instruments which incorporate laser technology in the treatment of benign prostatic hyperplasia, commonly referred to as BPH. BPH is a condition of an enlarged prostate gland, in which the gland having BPH typically increases in size to between about two to four times from normal. The lasers which are employed by the surgeons to treat this condition must have durable optical fibers that distribute light to the tissue to be treated in a predictable and controlled manner, and must also be capable of bending without breaking.

Lasers currently used for treating BPH typically employ one of two treatment modalities. The first modality is tissue ablation through surface absorption of laser energy by urethral and prostatic tissue, sometimes delivered by a side-firing laser device. In this modality, the laser wavelength can be selected to minimize the depth of penetration, e.g., typically shorter wavelengths in the visible spectrum.

A second modality is tissue coagulation through interstitial introduction of a diffuser fiberoptic. In this modality, the laser wavelength can be chosen to optimally penetrate the tissue to be treated. The optimal wavelength has typically been in the near-infrared spectrum, for example, around 830 nm. The targeted tissue is not ablated, but is necrosed through maintenance of a permanently damaging temperature of a volume of tissue adjacent the fiber. The body absorbs the necrosed tissue and the prostate shrinks to fill the void over time.

During the course of such treatments, one important parameter is the temperature of the tissue being treated. It is generally accepted that tissue can be irreversibly damaged by producing a temperature of 57° C. for one second. In order to produce this temperature at the desired radius from the applicator, the core temperature of the treatment site must be at some higher temperature, as is dictated by power deposition by the radiation, and thermal conduction from the deposition region. The core temperature is typically chosen to provide desired lesion size without producing tissue ablation at the applicator tip. For example, a current recommendation for forming lesions in the prostate as a treatment for BPH is to heat a small volume of tissue with a core target tissue temperature of 85° C., for approximately one and a half to three minutes. It can be appreciated that the size of the lesion formed is related to a combination of temperature and time, and the ability to reach a target temperature is related to the laser penetration, which is related to the laser wavelength, and the laser power level. Heating the tissue to lower temperatures for the same amount of time has the effect of incomplete lesion formation, while heating the tissue to significantly higher temperatures may ablate the tissue, cause excessive tissue damage and/or possible fiber material failure.

In general, more power is deposited in the tissue immediately adjacent the interstitial applicator, and thus this region generally reaches the highest treatment temperatures. In order to prevent ablation or tissue char, the highest temperatures should be maintained below 100° C. (e.g., 85° C.). Having a specified peak temperature for the treatment lesion, this temperature being typically located at the applicator, the resultant size of the lesion is dictated by the penetration depth of the treatment radiation. If the absorption is too high at the applicator tip, or the power deposited is too high due to large absorption, the peak acceptable temperature may be surpassed, causing non-optimal lesion, tissue ablation, and/or damage to the applicator. As stated previously, an example of an optimal wavelength that optimizes the treatment is in the wavelength region of the near infrared, for example, 830 nm. However, blood has an absorption in this region that may be considered non-optimal. If blood is present in the treatment region, the temperature of the lesion core will typically be higher for a given nominal treatment power than if the blood were not present. One method for mitigating this effect is to control the treatment temperature at the applicator tip, and adjusting treatment power to maintain the specified treatment temperature. If the absorption is high due to the presence of blood, the resultant treatment powers will be lower, and thus the lesion size may be lower than desired.

Controlling the temperature for the treatment has other desirable therapeutic effects. These include producing consistent lesion size despite varying physiologic characteristics, including perfusion rates and organ geometries, tissue absorption variations, and so on.

There are several ways of performing the temperature monitoring function for a laser system. One approach that has been utilized in laser treatment systems is known as the “Indigo 830e Laseroptic Treatment System” manufactured by Ethicon EndoSurgery, Inc. of Cincinnati, Ohio. This approach involves relying upon the temperature dependence of the fluorescent response of a slug of material at the fiber tip to an optical stimulus. More specifically, a pulse of pump energy causes a fluorescence pulse in an alexandrite slug which is delayed by a time interval corresponding to a temperature of the material. By providing the stimulus signal in the form of a sinusoid, the response signal is likewise a sinusoid and the temperature is related to the phase shift or difference therebetween.

Additionally, in the process of inserting the optical fiber through a patient's urethra and into the prostate, capillaries are sometimes broken and blood can be introduced alongside the fiber, between the fiber and the prostatic tissue. Hemoglobin (Hb) in blood is absorptive to near-infrared wavelengths, and at higher flux densities, the hemoglobin may absorb a large percentage of the laser energy near the fiber's surface. This absorption by the hemoglobin can increase the temperature near the fiber, which can damage the fiber as previously described. To avoid such fiber damage, the combination of energy flux and treatment temperature can be held below a certain pre-selected temperature and an infrared sensing system can be employed to stop treatment in the event that such damage is sensed.

### SUMMARY

In an aspect, a method for treating benign prostatic hyperplasia using a laser is provided. The method includes emitting, in proximity to prostatic tissue, laser light at a wavelength that is controlled to be within at least one of (i) a range between about 1275 nm and about 1475 nm or (ii) a range between about 1830 nm and about 2010 nm. The wavelength is selected to have a higher absorption by water than laser light at a wavelength of 830 nm and a lower absorption by hemoglobin than laser light at the wavelength of 830 nm. Emission of the laser light is controlled such that the prostatic tissue is heated to a temperature of less than about 100° C. to coagulate the prostatic tissue.

In another aspect, a laser system for coagulating prostatic tissue for treating benign prostatic hyperplasia is provided. The laser system includes a laser source configured to provide a laser beam having a wavelength that is within at least one of (i) a range between about 1275 nm and about 1475 nm or (ii) a range between about 1830 nm and about 2010 nm. The wavelength is selected to have a higher absorption by water than laser light at a wavelength of 830 nm and a lower absorption by hemoglobin than laser light at the wavelength of 830 nm. An optical fiber has a first end in optical communication with said laser source and a second end through which said laser beam is transmitted. A processor is included that control a power output from the laser so as to maintain a temperature of the optical fiber second end at a temperature of less than about 100° C.

The details of one or more embodiments of the invention are set forth in the accompanying drawings and the description below. Other features, objects, and advantages of the invention will be apparent from the description and the drawings, and from the claims.

### BRIEF DESCRIPTION OF THE DRAWINGS

FIG. 1 is a diagrammatic view of an embodiment of a medical device;

FIG. 2 illustrates a diagrammatic, perspective view of an embodiment of an optical fiber assembly;

FIG. 3 is a section view of an embodiment of a diffusive tip assembly for use with the medical device of FIG. 1;

FIG. 4 is a diagrammatic, detail illustration of the medical device of FIG. 1 inserted into prostatic tissue;

FIG. 5 is a plot of absorption coefficient for hemoglobin, both oxygenated and reduced hemoglobin, versus laser wavelength;

FIG. 6 is a plot of absorption coefficient for water versus laser wavelength;

FIG. 7 is μₐ for water, which dominates the absorption of human prostate tissue, in the region of the near infrared;

FIG. 8 is μ_{eff} for the case of human prostate tissue, in the region of the near infrared; and

FIG. 9 is an illustrative, detail view of a plot of absorption coefficient for water versus laser wavelength for a wavelength range around an absorption peak.

### DETAILED DESCRIPTION

As used herein, the term “proximal” refers to a location on a medical device 10 or a component thereof that is closer to a source of light energy and the term “distal” refers to a location on the medical device or a component thereof that is further from the source of light energy. Typically, the source of light energy of the medical device 10 is located outside a patient's body and the distal end of the medical device is insertable into the patient's body for a surgical procedure.

FIG. 1 shows an exemplary medical device 10 for diffusing light from an optical fiber 12. The medical device 10 includes the source of light energy 14, preferably a laser (e.g., a diode laser, a Er:YAG laser, Nd:YAG laser, diode-pumped tunable lasers, etc.). As will be described below, the source of light energy is capable of providing laser light at a wavelength within selected ranges that correspond to a desired absorption by water such as at wavelengths greater than about 1000 nm, such as between about 1000 nm and 3100 nm (e.g., at wavelengths having absorption coefficient of about 1.0 cm⁻¹, 5 cm⁻¹ or more, 20 cm⁻¹ or more, 50 cm⁻¹ or more). In some embodiments, the wavelength may be chosen to have an absorption coefficient value of approximately 1.5 cm⁻¹, for example, from about 1250 nm to 1400 nm, for example, at about 1325 nm. In some embodiments, the wavelength may be selected to correspond to water absorption peaks where the laser light is more readily absorbed by water. In particular, laser light may be provided at a wavelength within the infrared spectrum between about 1275 nm and about 1475 nm, between about 1830 nm and about 2010 nm, or between about 2950 nm and about 3050 nm.

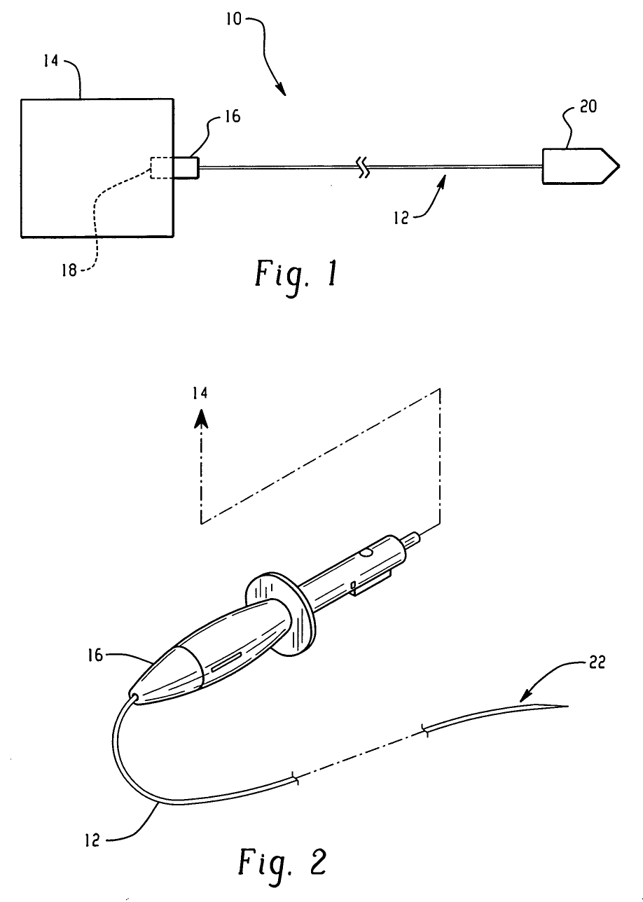

The optical fiber 12 is connected to the source of light energy 14 through an intermediary connector 16 at the proximal end of the fiber, which is attached to a connection port 18 of the source. A diffuser portion 20 is provided at the distal end of the optical fiber 12. An exemplary connector 16 and connection port 18 are described in U.S. Pat. No. 5,802,229 issued to Evans et al., the details of which are hereby incorporated by reference as if fully set forth herein. In some embodiments, the optical fiber 12 is provided and sold separately from the source of light energy 14, as an optical fiber assembly 22, as represented by FIG. 2.

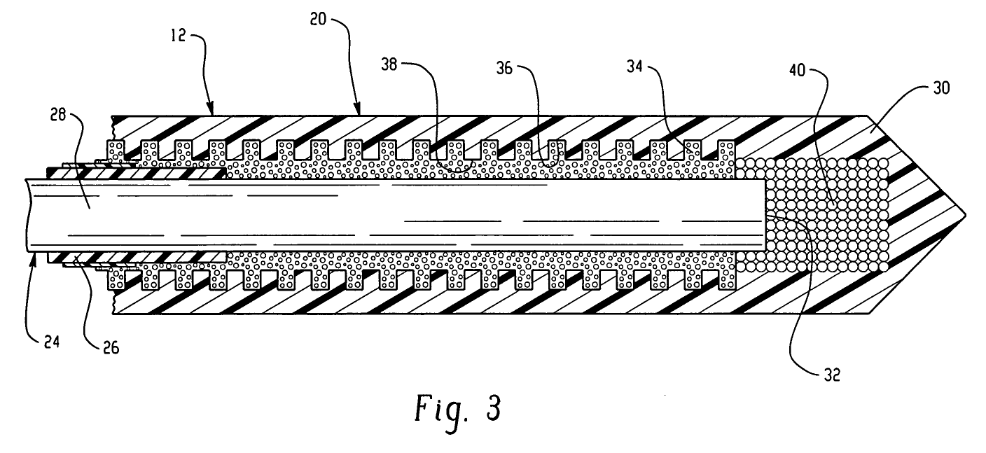

Referring now to FIG. 3, optical fiber 12 includes diffuser portion 20 and a light transmitting portion 24. At the light transmitting portion 24, a cladding 26 surrounds the core 28. In some embodiments, a sleeve (not shown) may also surround the cladding 26 and the core 28. Core 28 may be formed, for example, of silica glass, liquid or other materials that transmit laser energy at the wavelengths of interest with low loss. The material used to form the cladding 26 has an index of refraction that is lower than an index of refraction of the core 28 material so as to contain the light within the core. Cladding 26 terminates at a proximal end of a diffuser tip 30 and the core 28 extends into the diffuser tip of the diffuser portion 20 and terminates at a distal end 32. Diffuser tip 30 may be composed of a material that is flexible, is non-absorbent of laser energy in the wavelengths of interest, has a high melt temperature and is optically diffusing. Suitable materials for forming the diffuser tip 30 include perfluoroalkoxy (PFA) impregnated with barium sulfate, where the barium sulfate assists in scattering light energy, ethylenetetraflouroethylene (ETFE) and other types of fluoropolymers.

The distal portion of the core 28 extending into the diffuser tip 30 is used to diffuse light and is surrounded by an optical coupling material 34 at least partially disposed within a series of light directing features 36 that extend outwardly relative to a central, longitudinal axis of the diffuser tip 30. The optical coupling material 34 is a material having an index of refraction that is higher than the index of refraction of the core 28. Any suitable optical coupling material may be employed, such as XE5844 Silicone, which is made by General Electric Company; UV50 Adhesive, available from Chemence, Incorporated in Alpharetta, Ga.; and, 144-M medical adhesive, which is available from Dymax of Torrington, Conn.

A light-scattering component 40, which is filled with a light-scattering material and located at a distal face 42 of the core 28, can reflect light back into the core so as to provide a more even or uniform light distribution. Alexandrite, for example, can be employed as a light-scattering material for component 40. In addition to its light-scattering properties, the light-scattering component 40 material can fluoresce in a temperature-dependent manner upon being stimulated by light, with this property adapted to be used to measure temperature in tissue in proximity to the diffuser tip 30. Optical coupling adhesive, such as that described above, can be used to suspend the alexandrite particles therein and can serve as the base material for the light-scattering component 40. A method of forming various optical fiber 12 components including a light scattering component 40 can be found in U.S. Pat. No. 6,718,089 issued to James, I V et al., the details of which are hereby incorporated by reference as if fully set forth herein. Additional details of the exemplary optical fiber 12 is described in U.S. patent application Ser. No. 10/741,393, entitled “Optical Fiber Tip Diffuser and Method of Making Same”, filed Dec. 19, 2003, the details of which are hereby incorporated by reference as if fully set forth herein. Methods for measuring and controlling temperature of an optical fiber, for example, using a processor to control a power output from the laser are disclosed in U.S. patent application Ser. No. 10/650,535, entitled “System and Method of Measuring and Controlling Temperature of Optical Fiber Tip in a Laser System”, filed Aug. 28, 2003, the details of which are hereby incorporated by reference as if fully set forth herein.

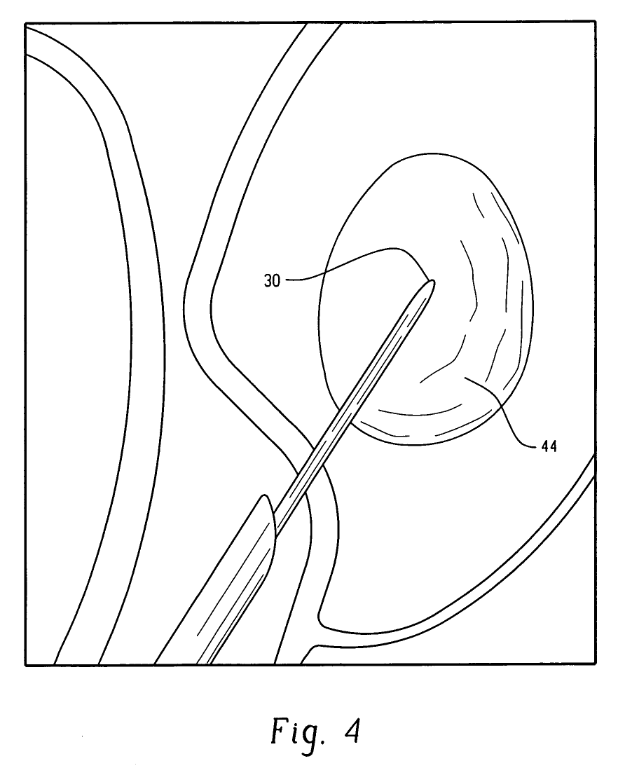

Referring to FIG. 4, during operation diffuser tip 30 is introduced to a patient's body through a cystoscope and inserted into prostatic tissue 44 in a fashion similar to that of a typical Interstitial Laser Coagulation (ILC) procedure. This insertion of the diffuser tip 30 into the prostatic tissue 44 can cause capillaries to break and blood to flow into a small gap between the diffuser tip 30 and the tissue.

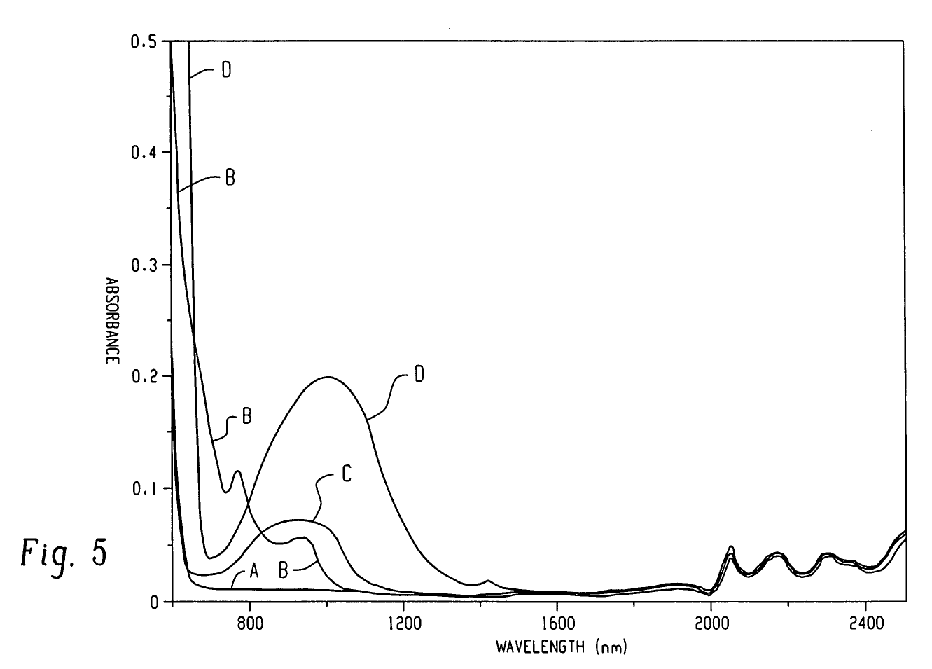

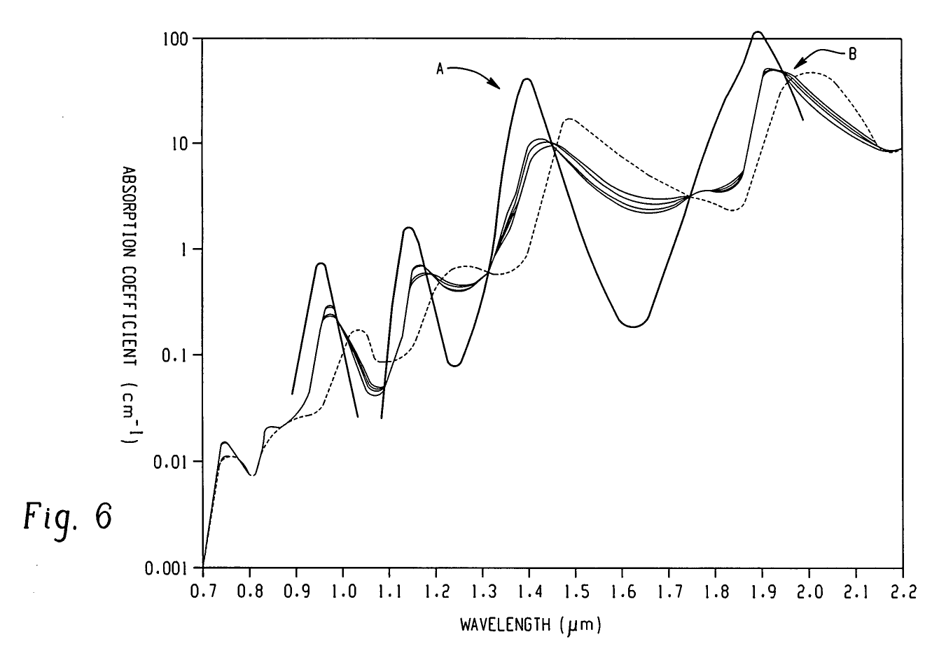

Referring to FIGS. 5 and 6, it is expected that use of laser wavelengths having a lower absorption by hemoglobin and an optimal higher absorbance by water may provide advantages, particularly over those wavelengths that are readily absorbed by hemoglobin and readily transmitted by water, such as at wavelengths around 830 nm. In particular, it is expected that use of laser wavelengths more readily absorbed by water and less readily absorbed by hemoglobin will allow for use of flux densities up to about 70 W/cm² with little additional concern for tissue charring or fiber 12 material damage caused by laser energy absorption by hemoglobin at the fiber surface. Without flux limits imposed by hemoglobin surface absorption, greater flux density will bring the volume of tissue under treatment to the target treatment temperature faster (e.g., 100° C. or less, such as between about 85° C. and 100° C.), which can result in shorter treatment times, a benefit to both physician and patient.

Additionally, it may be advantageous to adjust the penetration depth, either a priori, or during treatment, in order to match the lesion size to the targeted tissue or organ, to maximize the lesion size or to otherwise produce a particular size of lesion. If the wavelength is adjusted during the treatment in order to adjust the penetration depth, this could be in response to feedback from a sensor or feedback sensor system. This might include a temperature sensing system, as already described, from a sensor on the applicator, a sensor located separately from the applicator or a sensor detecting the characteristic blackbody radiation of the treatment site, for example through the treatment fiber. The sensor or sensing system might detect tissue optical characteristics, such as scatter, or mechanical properties such as modulus, or other characteristics, such as water content, elasticity or conductivity.

It is generally desirable to match the lesion size to the target organ or targeted tissue. In the case of BPH, the prostate is typically 2-3 cm in radius, and generally ellipsoidal approximating spherical in shape. A radiation penetration depth that is too small results in lesions sizes that may not produce a clinically useful treatment. Penetration depths that are too large can heat tissue beyond the boundary of the targeted organ or tissue.

The absorption characteristic of the radiation in the target tissue depends primarily on three phenomena: the native absorption of the photons in the tissue (μₐ), the scatter of the photons in the tissue (μₐ) and the scatter angle (g) through which the photon is scattered. Typically, the scatter coefficient and angle are incorporated into one parameter, the “reduced scatter coefficient,”

μ′ₛ=μₛ(1−g).

### The effective absorption coefficient may then be approximated by

μ_{eff}=(3μₐ(μₐ+μ′ₛ))^{0.5}.

The native absorption coefficient (μₐ) is affected by the molecular absorption characteristic of the tissue constituents being irradiated. In general, the scatter characteristic in tissue reduces as wavelength increases. The tissue scatter is dependent on the structure of the tissues being irradiated. The structure is constant, and thus the scatter coefficient is generally a smoothly varying value that decreases with longer wavelengths.

FIG. 5 shows the absorbance spectrum of species of hemoglobin. Absorbance is a measure of absorption per unit depth of penetration into a material. Line A is carboxyhemoglobin, line B is deoxyhemoglobin, line C is oxyhemoglobin and D is methemoglobin. The absorbance of the hemoglobin species methemoglobin D, deoxyhemoglobin B and oxyhemoglobin C are lower at certain wavelengths above 1000 nm than at 830 nm.

FIG. 6 shows a plot of the absorption coefficient of water as a function of laser wavelength. The spectral absorption coefficient is a measure of how well a material absorbs light at particular wavelengths. As can be seen, water has several absorption bands or peaks A and B at certain wavelengths above 1000 nm, in particular, at ranges within the infrared spectrum between about 1275 nm and about 1475 nm (e.g., between about 1420 nm and about 1460 nm, such as at about 1440), between about 1830 nm and about 2010 nm (e.g., between about 1910 nm and about 1950 nm, such as at about 1930), and between about 2950 nm and about 3050 nm (e.g., between about 2080 nm and about 3020 nm, such as at about 3000 nm).

There are also intermediate spectral regions in the water absorption spectra where absorption is relatively low. In the region from 1000 nm to 2000 nm, the water absorption has values from less than about 0.1/cm to about 100/cm, in other words, values over three orders of magnitude. In these wavelength ranges, the hemoglobin absorption coefficient is much lower than the hemoglobin absorption coefficient at 830 nm. During use in treating BPH, the relative low hemoglobin absorption coefficient can provide the advantages described above.

The optical properties of the human prostate are known at some wavelengths. For example, at a wavelength 633 nm, μ′ₛ is about 8.6/cm, μₐ is about 0.7/cm and the resultant μ_{eff} is about 4.4/cm. At a wavelength of 1064 nm, μ′ₛ is about 6.4/cm, μₐ is about 1.5/cm and μ_{eff} is about 5.9/cm. At a wavelength of 830 nm, μ_{eff} is about 4/cm to about 5/cm. At a wavelength of 1325 nm, the reduced scatter characteristic (μ′ₛ) would be expected to be about 4/cm and the absorption of the prostate tissue will be dominated by that of water, which has a μₐ of about 1.5/cm, yielding a μ_{eff} of about 4/cm to about 5/cm.

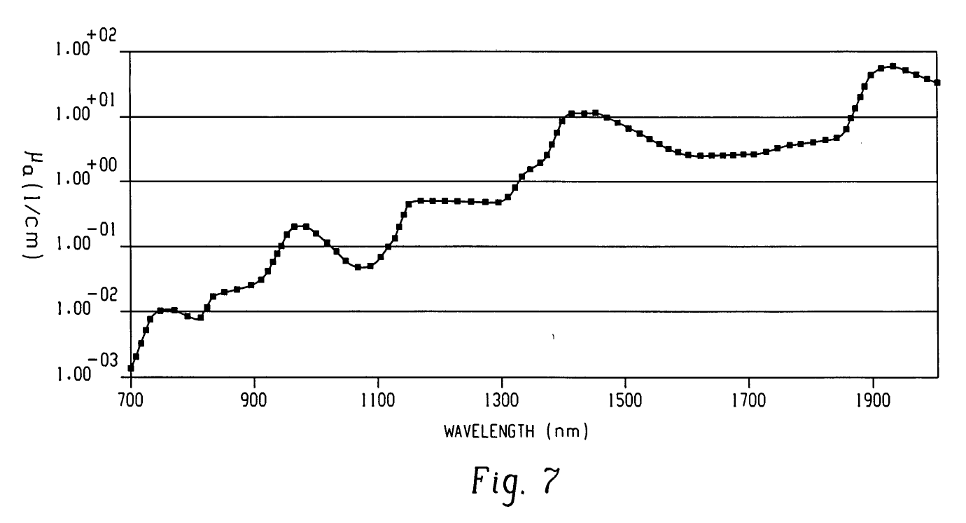

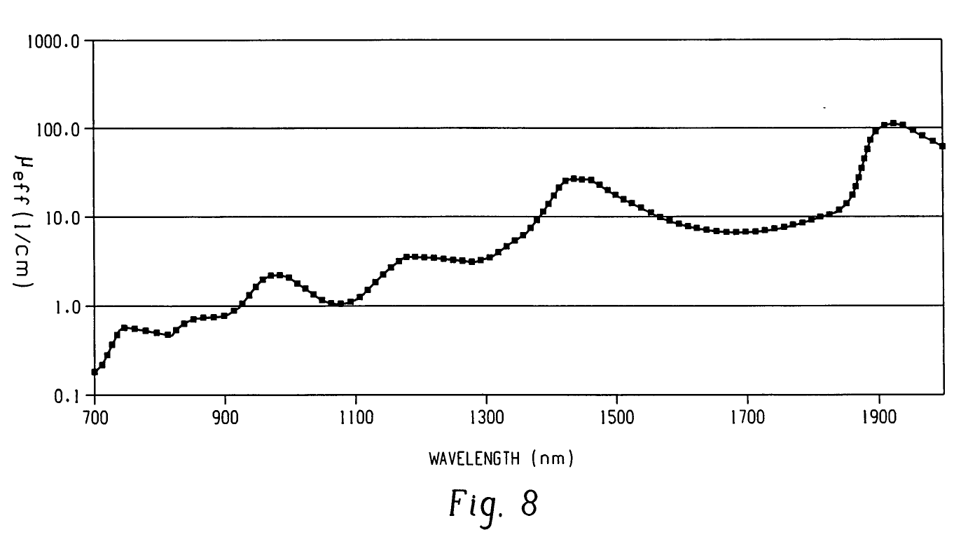

Referring to FIGS. 7 and 8, the values of μ_{eff} span a wide range of values, and a wide variety of desired values can be obtained with wavelength selection, or wavelength tuning of an optical source. It would be desirable to produce an absorption in the prostate tissue similar to that at 830 nm at a wavelength range where blood has little absorption. This would facilitate avoiding potential negative factors associated with an undue amount of blood that sometimes may be present, as previously discussed, while maintaining an absorption that well matches the prostate, also previously discussed. In some embodiments, a μ_{eff} of the prostate tissue equal to that at about 830 nm may be produced by the application of light energy at approximately 1325 nm, where μₐ is about 1.5/cm and μ_{eff} is about 4/cm to about 5/cm. At this wavelength of about 1325 nm, the radiation energy will experience a small absorption in hemoglobin.

Advantageously, tunable sources of optical radiation are readily available in the wavelength range of 1300 nm, or if a higher absorption by water is desired (and thereby the prostate tissue), in the region of 1550 nm, due to these sources' utility and pervasiveness in the fiber optic communications industry. Additionally, the penetration radiation may be readily manipulated by adjusting the wavelength around 1325 nm where water has a rapidly changing absorption. Thus, the penetration depth may be readily adjusted to manipulate resultant lesion size. The may be done, for example, to maximize lesion size, to minimize treatment time for a given lesion size or to adjust a lesion size for a given target or organ.

Additional advantages may be realized. For example, as tissue treatment progresses and the tissue becomes denatured closer to the fiber, absorption of the prostate tissue near the fiber will likely decrease due to the lack of water, causing the laser energy to move further away from the fiber before being absorbed by fresh tissue. Thus, a self-limiting treatment may be provided since as the treatment volume increases, the laser energy decreases with penetration distance. Eventually, in some instances, the energy density may decrease to the point where the tissue is merely heated without permanent consequences.

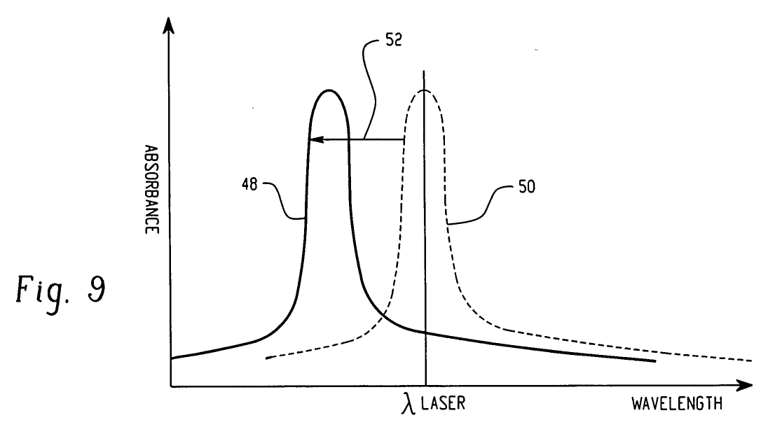

It may be desirable to utilize the temperature dependent shift of the characteristic peak features of water absorption (e.g., at 1440 nm, 1930 nm and 3000 nm) in order to achieve a desirable absorption change as temperature increases. Laser wavelength can be chosen specifically for a high negative value of d(mu)/dT, thereby equalizing the temperature field “automatically” against variable such as local optical field and blood flow variations. Referring to FIG. 9, an illustrative view of an absorption coefficient line 48 for water at a water absorption peak is shown. As an additional self-limiting treatment feature, the absorption line “blue shifts” in the direction of arrow 52 or peaks at a shorter wavelength as the water temperature increases. Due to the spectral narrowness of the peak of the absorption coefficient line, this results in a decrease in laser energy being absorbed by the water at the higher temperature with a corresponding increase in depth of penetration of the laser energy. The resulting benefit is that tissue nearest the fiber will be less likely to be over-treated. Dotted line 50 represents the absorbance for water at body temperature while line 48 represents a blue shifted absorption line for water at the treatment temperature.

In the vicinity of 1300 nm, the temperature dependent change of absorption is different at wavelengths lower than 1300 nm, where the absorption decreases with increased temperature compared to wavelengths longer than 1300 nm, where the absorption increases with increased temperature. In some embodiments, laser wavelengths may be chosen at the minima of the derivative spectrum of water absorption with respect to temperature.

In some embodiments, temperature dependent water absorption may be used to deduce temperature near the medical device for example by monitoring back-scattered light at the laser wavelength and a nearby wavelength for which water absorption is not temperature dependent. The ratio of backscattered light at these wavelengths can specify local tissue temperature, whereas changes in the non-temperature dependent wavelength can independently monitor tissue scattering changes during thermal coagulation.

The above-described system and method of treating BPH can provide several advantages over known BPH treatments. By irradiating prostatic tissue at wavelengths that are more readily absorbed by water and are less readily absorbed by hemoglobin, greater flux densities can be utilized with less additional concern for material damage and overtreatment of tissue.

A number of detailed embodiments have been described. Nevertheless, it will be understood that various modifications may be made. Accordingly, other embodiments are within the scope of the following claims.

What is claimed is:

1. A method for treating benign prostatic hyperplasia using a laser, the method comprising:
emitting, in proximity to prostatic tissue, laser light at a wavelength that is controlled to be within at least one of (i) a range between about 1275 nm and about 1475 nm or (ii) a range between about 1830 nm and about 2010 nm, the wavelength selected to have a higher absorption by water than laser light at a wavelength of 830 nm and a lower absorption by hemoglobin than laser light at the wavelength of 830 nm; and
controlling emission of the laser light such that the prostatic tissue is heated to a temperature of less than about 100° C. to coagulate the prostatic tissue.

2. The method of claim 1, wherein the wavelength of light emitted in proximity to the prostatic tissue is between about 1275 nm and about 1325 nm.

3. The method of claim 1, wherein the wavelength of light emitted in proximity to the prostatic tissue is about 1325 nm.

4. The method of claim 1 further comprising tuning the wavelength in response to a change in property of the prostatic tissue, wherein the property is at least one of temperature, absorption, scatter, or a thermo-mechanical property of the prostatic tissue.

5. The method of claim 1 further comprising tuning the wavelength to produce a desired lesion.

6. The method of claim 1 further comprising
introducing an optical fiber into a patient's body;
locating a light-diffusing tip of the optical fiber adjacent the prostatic tissue; and
the optical fiber transmitting laser light from a source of light energy to the light-diffusing tip.

7. The method of claim 6 further comprising decreasing absorbance of laser energy by the prostatic tissue by denaturing the prostatic tissue using the laser light as the prostatic tissue near the light-diffusing tip is treated.

8. The method of claim 6 further comprising decreasing the absorbance of laser energy by the prostatic tissue by a thermally-induced decrease in absorbance by the tissue being treated at the wavelength.

9. The method of claim 8 further comprising determining temperature near the light diffusing tip by monitoring back-scattered light at the wavelength and another wavelength for which water absorption is not temperature dependent.

10. The method of claim 1, wherein, in the step of controlling, the prostatic tissue is heated to a temperature of between about 85° C. and about 100° C. to coagulate the prostatic tissue.

11. A laser system for coagulating prostatic tissue for treating benign prostatic hyperplasia, the laser system comprising:
a laser source configured to provide a laser beam having a wavelength that is within at least one of (i) a range between about 1275 nm and about 1475 nm or (ii) a range between about 1830 nm and about 2010 nm, the wavelength selected to have a higher absorption by water than laser light at a wavelength of 830 nm and a lower absorption by hemoglobin than laser light at the wavelength of 830 nm;
an optical fiber having a first end in optical communication with said laser source and a second end through which said laser beam is transmitted; and
a processor that controls a power output from the laser so as to maintain a temperature of the optical fiber second end at a temperature of less than about 100° C.

12. The laser system of claim 11, wherein the wavelength of the laser beam is between about 1275 nm and about 1325 nm.

13. The laser system of claim 12, wherein the wavelength of the laser beam is about 1325 nm.

14. The laser system of claim 11, wherein the laser source is configured to be wavelength tunable such that the wavelength can be tuned in response to change in property of treated tissue.

15. The laser system of claim 11, wherein the processor controls a power output from the laser so as to maintain a temperature of the optical fiber second end at a temperature of between about 85° C. and about 100° C.

16. The laser system of claim 11 further comprising a diffuser tip located at a distal end of the optical fiber for diffusing the laser beam.
